SEQUENCE LISTING (13 sequences)

SEQ ID NO: 1 - protein, 14 aa, Artificial Sequence
  Synthetically generated sequence
CCEFCCNPACTGCY

SEQ ID NO: 2 - protein, 13 aa, Artificial Sequence
  Synthetically generated peptide
CCEFCCNPACTGC

SEQ ID NO: 3 - protein, 14 aa, Artificial Sequence
  Synthetically generated peptide
CCEICCNPACTGCY

SEQ ID NO: 4 - protein, 13 aa, Artificial Sequence
  Synthetically generated peptide
CCEICCNPACTGC

SEQ ID NO: 5 - protein, 14 aa, Artificial Sequence
  Synthetically generated peptide
CCELCCNPACTGCY

SEQ ID NO: 6 - protein, 13 aa, Artificial Sequence
  Synthetically generated peptide
CCELCCNPACTGC

SEQ ID NO: 7 - protein, 14 aa, Artificial Sequence
  Synthetically generated peptide
CCEWCCNPACTGCY

SEQ ID NO: 8 - protein, 13 aa, Artificial Sequence
  Synthetically generated peptide
CCEWCCNPACTGC

SEQ ID NO: 9 - protein, 13 aa, Artificial Sequence
  Synthetically generated peptide
CCEYCCNPACTGC

SEQ ID NO: 10 - protein, 15 aa, Artificial Sequence
  Synthetically generated peptide
PGTCEICAYAACTGC

SEQ ID NO: 11 - protein, 16 aa, Artificial Sequence
  Synthetically generated peptide
NDDCELCVNVACTGCL

SEQ ID NO: 12 - protein, 16 aa, Artificial Sequence
  Synthetically generated peptide
NDECELCVNVACTGCL

SEQ ID NO: 13 - protein, 14 aa, Artificial Sequence
  Synthetically generated peptide
CCEYCCNPACTGCY

(19) United States
(12) Patent Application Publication
(10) Pub. No.: US 2012/0039949 A1
(43) Pub. Date: Feb. 16, 2012

(54) Stable Solid Formulations of GC-C Receptor Agonist Polypeptides Suitable for Oral Administration

(75) Inventors: Angelika Fretzen, Somerville, MA (US); Steven Witowski, Melrose, MA (US); Alfredo Grossi, Somerville, MA (US); Hong Zhao, Somerville, MA (US)

(73) Assignee: Ironwood Pharmaceuticals, Inc., Cambridge, MA (US)

(21) Appl. No.: 13/062,324

(22) PCT Filed: Aug. 14, 2009
(86) PCT No.: PCT/US09/04685

§ 371 Date: Oct. 24, 2011

Related U.S. Application Data
(60) Provisional application No. 61/094,327, filed on Sep. 4, 2008.

(51) Int. Cl. A61K 9/00; A61P 29/00; A61P 1/00; A61K 38/02; A61K 38/10

(52) U.S. Cl. 424/400; 514/1.1; 514/21.5; 514/21.4

(57) ABSTRACT

Solid, stable formulations of GC-C receptor agonist polypeptide suitable for oral administration are described herein as are methods for preparing such formulations. The GC-C receptor agonist polypeptide formulations described herein are stable and have a sufficient shelf life for manufacturing, storing and distributing the drug.

### PRIORITY CLAIM

This application claims priority to U.S. application Ser. No. 61/094,327, filed Sep. 4, 2008. The entire contents of the aforementioned application are incorporated herein by reference.

### FIELD

This disclosure concerns solid formulations of a guanylate cyclase-C receptor agonist polypeptide suitable for oral administration and methods for preparing such formulations.

### BACKGROUND

Many therapeutic polypeptides are formulated in aqueous solution because they are most active in this form. However, most polypeptides are not particularly stable in aqueous solution, such that the formulations often have a short half-life and require refrigeration. Although aqueous solutions of polypeptides can be dried by freeze-drying, spray-drying or other methods, such dried formulations may also be unstable and have reduced activity relative to an aqueous solution of the polypeptide. Typical break-down mechanisms that occur both in aqueous solution and in dried formulations include aggregation and oxidative and/or hydrolytic degradation. Thus, the majority of therapeutic polypeptides, whether in aqueous solution or dried, are stored under refrigerated conditions due to their limited stability.

Polypeptides that activate guanylate cyclase-C (GC-C) receptor can be useful for treating gastrointestinal disorders and conditions, including irritable bowel syndrome (IBS) and chronic constipation (CC). Generally, formulations comprising polypeptides need to be refrigerated in order to avoid degradation over time. However, refrigeration is inconvenient both for commercial distribution of the drug and for storage by patients. Thus, it is desirable to have formulations that have increased stability at room temperature.

### SUMMARY

Solid, stable formulations of polypeptides that activate the GC-C receptor (GC-C receptor agonist polypeptide) suitable for oral administration are described herein as are methods for preparing such formulations. The formulations described herein contain a polypeptide comprising, consisting of or consisting essentially of an amino acid sequence as described herein. Polypeptides that may be useful include those disclosed in any of: WO 2004/069165, WO2005/087797, WO 2007/022531, WO2005/016244, WO2005/074575, WO2006/102069, WO2008/002971, WO2008/106429, WO 2008/137318, WO2002/078683, WO 2006/086653, WO 2007/101158, WO 2008/151257, U.S. Pat. No. 7,041,786, and WO 2007/101161. In addition polypeptides disclosed in US 20030073628, JP 2009001582, EP1379224, CA2441970AA, and US 20090048175 may also be useful.

In some embodiments, there is provided a pharmaceutical composition comprising a pharmaceutically acceptable carrier, GC-C receptor agonist polypeptide and one or more agents selected from Mg²⁺, Ca²⁺, Zn²⁺, Mn²⁺, K⁺, Na^{+or Al}³⁺or a sterically hindered primary amine, wherein the agent improves at least one attribute of the composition, relative to a pharmaceutical composition without the agent. In further embodiments, the agent is Mg²⁺, Ca²⁺ or Zn²⁺. In a further embodiment, the agent is Ca²⁺. In some embodiments, the cation is provided, without limitation, as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate. In further embodiments, the cation is provided as magnesium chloride, calcium chloride, calcium phosphate, calcium sulfate, zinc acetate, manganese chloride, potassium chloride, sodium chloride or aluminum chloride. In other embodiments, the cation is provided as calcium chloride, magnesium chloride or zinc acetate.

In another embodiment, the agent is a sterically hindered primary amine. In a further embodiment, the sterically hindered primary amine is an amino acid. In yet a further embodiment, the amino acid is a naturally-occurring amino acid. In a still further embodiment, the naturally-occurring amino acid is selected from the group consisting of: histidine, phenylalanine, alanine, glutamic acid, aspartic acid, glutamine, leucine, methionine, asparagine, tyrosine, threonine, isoleucine, tryptophan, glycine and valine; yet further, the naturally-occurring amino acid is leucine, isoleucine, alanine or methionine; in another embodiment, the naturally-occurring amino acid is leucine or methionine; still further, the naturally-occurring amino acid is leucine. In another embodiment, the sterically hindered primary amine is a non-naturally occurring amino acid (e.g., 1-aminocyclohexane carboxylic acid). In a further embodiment, the sterically hindered primary amine is cyclohexylamine, 2-methylbutylamine or chitosan. In further embodiments, the pharmaceutical composition comprising a GC-C receptor agonist polypeptide is a mixture of two or more GC-C receptor agonist polypeptides.

In other embodiments, there is provided a pharmaceutical composition comprising a pharmaceutically acceptable carrier, a GC-C receptor agonist polypeptide, a cation selected from Mg²⁺, Ca²⁺, Zn²⁺, Mn²⁺, Na⁺ or Al³⁺ and a sterically hindered primary amine. In one embodiment, the cation is Mg²⁺, Ca²⁺, Zn²⁺. In a further embodiment, the cation is Ca²⁺. In another embodiment, the cation is a mixture of two or three of Mg²⁺, Ca²⁺and Zn²⁺. In a further embodiment, the pharmaceutical composition further comprises a pharmaceutically acceptable binder and/or a pharmaceutically acceptable glidant, lubricant or additive that acts as both a glidant and lubricant and/or an antioxidant. In a further embodiment, the sterically hindered primary amine is an amino acid. In yet a further embodiment, the amino acid is a naturally-occurring amino acid. In a still further embodiment, the naturally-occurring amino acid is selected from the group consisting of: histidine, phenylalanine, alanine, glutamic acid, aspartic acid, glutamine, leucine, methionine, asparagine, tyrosine, threonine, isoleucine, tryptophan, glycine and valine; yet further, the naturally-occurring amino acid is leucine, isoleucine, alanine or methionine; in another embodiment, the naturally-occurring amino acid is leucine or methionine; still further, the naturally-occurring amino acid is leucine. In another embodiment, the sterically hindered primary amine is a non-naturally occurring amino acid (e.g., 1-aminocyclohexane carboxylic acid). In a further embodiment, the sterically hindered primary amine is cyclohexylamine, 2-methylbutylamine or chitosan. In another embodiment, the sterically hindered primary amine can be a mixture of more than one sterically hindered primary amine. For example, the sterically hindered primary amine may be a mixture of two or more amino acids.

In some cases the molar ratio of cation:sterically hindered primary amine:GC-C receptor agonist polypeptide (e.g., Ca²⁺:leucine:GC-C receptor agonist polypeptide) in the aqueous solution applied to the carrier is 5-100:5-50:1. It can be desirable for the molar ratio of cation:sterically hindered primary amine (e.g., Ca²⁺:leucine) to be equal to or greater than 2:1 (e.g., between 5:1 and 2:1). Thus, in some cases the molar ratio of cation:sterically hindered primary amine:GC-C receptor agonist polypeptide (e.g., Ca²⁺:leucine:GC-C receptor agonist polypeptide) applied to the carrier is 100:50:1, 100:30:1, 80:40:1, 80:30:1, 80:20:1, 60:30:1, 60:20:1, 50:30:1, 50:20:1, 40:20:1, 20:20:1, 10:10:1, 10:5:1 or 5:10:1. When binder, e.g., methylcellulose, is present in the GC-C receptor agonist polypeptide solution applied to the carrier it can be present at 0.5%-2.5% by weight (e.g., 0.7%-1.7% or 0.7%-1% or 1.5% or 0.7%).

The weight of GC-C receptor agonist polypeptide applied to a given weight of filler (e.g., microcrystalline cellulose) can vary from about 0.02:100 to about 2.67:100. Thus, about 0.05 mg to about 6.0 mg of GC-C receptor agonist polypeptide can be applied to 225 mg of filler. In a further embodiment, the weight of GC-C receptor agonist polypeptide applied to a given weight of filler is about 0.05 mg to about 2.0 mg of GC-C receptor agonist polypeptide (e.g., 0.1, 0.2, 0.3. 0.4, 0.5, 0.6, 0.7 mg peptide for 225 mg of filler).

In various embodiments: the sterically hindered primary amine is an amino acid (e.g., a naturally-occurring amino acid or a naturally-occurring amino acid selected from histidine, phenylalanine, alanine, glutamic acid, aspartic acid, glutamine, methionine, asparagine, tyrosine, threonine, leucine, isoleucine, tryptophan, or valine). In other cases the sterically hindered primary amine is a non-naturally occurring amino acid or amino acid derivative (e.g., lanthionine, theanine or 1-amino cyclohexane). In other cases, the sterically hindered primary amine is an amino sugar (e.g., chitosane or glucosamine).

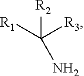

In some cases, the sterically hindered primary amine has the formula: wherein R₁, R₂ and R₃ are independently selected from: H; —C(O)OH; C1-C6 alkyl, optionally substituted by —CO₂H, —CONH₂, or a 5-10 membered aryl or heteroaryl; C1-C6 alkoxyalkyl; or C1-C6 thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

In various cases: the antioxidant is selected from BHA (butylated hydroxyanisole), BHT (butylated hydroxytoluene), vitamin E, propyl gallate, ascorbic acid and salts or esters thereof, tocopherol and esters thereof, alpha-lipoic acid, beta-carotene; the pharmaceutically acceptable binder is polyvinyl alcohol or polyvinyl pyrrolidone; the pharmaceutically acceptable binder is selected from: a starch (e.g., corn starch, pre-gelatinized potato starch, rice starch, wheat starch, and sodium starch glycollate), maltodextrin or a cellulose ether (e.g., methylcellulose, ethylcellulose, carboxymethylcellulose, hydroxyethyl cellulose, hydroxyethyl methylcellulose, hydroxypropyl cellulose and hydroxypropyl methyl cellulose); the pharmaceutically acceptable filler is cellulose (e.g., microfine cellulose or microcrystalline cellulose such as Celphere CP-305 or Avicel); the pharmaceutically acceptable filler is a sugar or a sugar alcohol (e.g., mannitol, isomalt, sorbitol, dextrose, xylitol, sucrose and lactose); the filler comprises particles having an average diameter between 50 mm and 1000 μm; the lubricant and/or glidant is selected from: talc, leucine, magnesium stearate, stearic acid and polyvinyl alcohol; and the lubricant and/or glidant is selected from: calcium stearate, mineral oil, vegetable oil, polyethylene glycol (PEG e.g., PEG that is liquid or solid at room temperature), sodium benzoate, and sodium lauryl sulfate.

In some cases, the GC-C receptor agonist polypeptide solution used in a method for preparing the formulation has a pH below 7 (e.g., a pH between 1 and 3 or a pH between about 1.5 and about 2.5). The pH can be adjusted with, e.g., phosphoric acid. In some cases, the solution is buffered. Various pharmaceutically acceptable buffers can be used (e.g., phosphate buffer).

In some cases, the GC-C receptor agonist polypeptide solution used in a method for preparing the formulation comprises both a cation (e.g., CaCl₂) and a sterically hindered primary amine (e.g., leucine).

In some cases the GC-C receptor agonist polypeptide solution comprises CaCl₂ and leucine; the binder is methylcellulose; the filler is microcrystalline cellulose; the glidant and/or lubricant comprises talc or leucine. In further embodiments, the pharmaceutical composition comprising a GC-C receptor agonist polypeptide is a mixture of two or more GC-C receptor agonist polypeptides.

Also provided is a pharmaceutical composition prepared by any of the methods described herein.

The GC-C receptor agonist polypeptide formulations described herein can be stable and can have a sufficient shelf life for manufacturing, storing and distributing the drug. For example, formulations described herein are expected to have a shelf life of at least 12 months at room temperature storage conditions (e.g., 25° C./60% relative humidity (RH)). In further embodiments, the formulations described herein are expected to have a shelf life of at least 18 months or at least 24 months at room temperature storage conditions (e.g., 25° C./60% RH). Thus, when assessed in an assay on a weight/weight basis as determined by high pressure liquid chromatography (HPLC) against a GC-C receptor agonist polypeptide reference standard, ≧95% of the original amount of GC-C receptor agonist polypeptide in the composition remains after three months when packaged samples are stored at accelerated conditions (40° C./75% RH). In further embodiments, ≧90% of the original amount of GC-C receptor agonist polypeptide in the composition remains after at least 6 months when packaged samples are stored at accelerated conditions (40° C./75% RH). In addition, chromatographic purity of the GC-C receptor agonist polypeptide as determined as area percent by HPLC remains at ≧90% over the course of at least three months when packaged samples are stored at accelerated conditions (40° C./75% RH). In further embodiments, the chromatographic purity of the GC-C receptor agonist polypeptide as determined by area percent by HPLC remains at ≧90% over the course of at least 6 months when packaged samples are stored at accelerated conditions (40 ° C./75% RH). Thus, for example, no more than about 10% of the GC-C receptor agonist polypeptide undergoes degradation to other products.

In one embodiment, the invention comprises a pharmaceutical composition comprising GC-C receptor agonist polypeptide, wherein the chromatographic purity of the GC-C receptor agonist polypeptide decreases by less than 15% or decreases by less than 10% after 18 months or 24 months of storage of the pharmaceutical composition at 25° C. at 60% relative humidity in a sealed container containing a desiccant. In a further embodiment, the chromatographic purity of the GC-C receptor agonist polypeptide decreases by less than 9%, 8%, 7%, 6%, 5%, 4% or 2% after 18 months or 24 months of storage of the pharmaceutical composition at 25° C. at 60% relative humidity in a sealed container containing a desiccant. In another embodiment, the invention comprises a pharmaceutical composition comprising GC-C receptor agonist polypeptide, wherein the chromatographic purity of the GC-C receptor agonist polypeptide decreases by less than 15% or decreases by less than 10% after 3 months or 6 months of storage of the pharmaceutical composition at 40° C. at 75% relative humidity in a sealed container containing a desiccant. In a further embodiment, the chromatographic purity of the GC-C receptor agonist polypeptide decreases by less than 9%, 8%, 7%, 6%, 5%, 4% or 2% after 3 months or 6 months of storage of the pharmaceutical composition at 40° C. at 75% relative humidity in a sealed container containing a desiccant. In one embodiment, the invention comprises a unit dosage form of a pharmaceutical composition comprising GC-C receptor agonist polypeptide, wherein the chromatographic purity of the GC-C receptor agonist polypeptide decreases by less than 15% or decreases by less than 10% after 18 months or 24 months of storage of the unit dosage form at 25° C. at 60% relative humidity in a sealed container containing a desiccant. In a further embodiment, the chromatographic purity of the GC-C receptor agonist polypeptide decreases by less than 9%, 8%, 7%, 6%, 5%, 4% or 2% after 18 months or 24 months of storage of the unit dosage form at 25° C. at 60% relative humidity in a sealed container containing a desiccant. In another embodiment, the invention comprises a unit dosage form of a pharmaceutical composition comprising GC-C receptor agonist polypeptide, wherein the chromatographic purity of the GC-C receptor agonist polypeptide decreases by less than 15% or decreases by less than 10% after 3 months or 6 months of storage of the unit dosage form at 40° C. at 75% relative humidity in a sealed container containing a desiccant. In a further embodiment, the chromatographic purity of the GC-C receptor agonist polypeptide decreases by less than 9%, 8%, 7%, 6%, 5%, 4% or 2% after 3 months or 6 months of storage of the unit dosage form at 40° C. at 75% relative humidity in a sealed container containing a desiccant.

In one embodiment, the invention comprises a sealed container comprising a plurality of unit dosage forms of a pharmaceutical composition comprising GC-C receptor agonist polypeptide, wherein the chromatographic purity of the GC-C receptor agonist polypeptide decreases by less than 15% or decreases by less than 10% after 18 months or 24 months of storage of the sealed container containing a desiccant at 25° C. at 60% relative humidity. In a further embodiment, the chromatographic purity of the GC-C receptor agonist polypeptide decreases by less than 9%, 8%, 7%, 6%, 5%, 4% or 2% after 18 months or 24 months of storage of the sealed container containing a desiccant at 25° C. at 60% relative humidity. In another embodiment, the invention comprises a sealed container comprising a plurality of unit dosage forms of a pharmaceutical composition comprising GC-C receptor agonist polypeptide, wherein the chromatographic purity of the GC-C receptor agonist polypeptide decreases by less than 15% or decreases by less than 10% after 3 months or 6 months of storage of the sealed container containing a desiccant at 40° C. at 75% relative humidity. In a further embodiment, the chromatographic purity of the GC-C receptor agonist polypeptide decreases by less than 9%, 8%, 7%, 6%, 5%, 4% or 2% after 3 months or 6 months of storage of the sealed container containing a desiccant at 40° C. at 75% relative humidity.

In one embodiment, the invention comprises a pharmaceutical composition comprising GC-C receptor agonist polypeptide, wherein the assay value for GC-C receptor agonist polypeptide determined on a weight/weight basis decreases by less than 15% or decreases by less than 10% after 18 months or 24 months of storage of the pharmaceutical composition at 25° C. at 60% relative humidity in a sealed container containing a desiccant. In a further embodiment, the assay value for GC-C receptor agonist polypeptide determined on a weight/weight basis decreases by less than 9%, 8%, 7%, 6%, 5%, 4%, 3%, 2% or 1% after 18 months or 24 months of storage of the pharmaceutical composition at 25° C. at 60% relative humidity in a sealed container containing a desiccant. In another embodiment, the invention comprises a pharmaceutical composition comprising GC-C receptor agonist polypeptide, wherein the assay value for GC-C receptor agonist polypeptide determined on a weight/weight basis decreases by less than 15% or decreases by less than 10% after 3 months or 6 months of storage of the pharmaceutical composition at 40° C. at 75% relative humidity in a sealed container containing a desiccant. In a further embodiment, the chromatographic purity of the GC-C receptor agonist polypeptide decreases by less than 9%, 8%, 7%, 6%, 5%, 4%, 3%, 2% or 1% after 3 months or 6 months of storage of the pharmaceutical composition at 40° C. at 75% relative humidity in a sealed container containing a desiccant.

In one embodiment, the invention comprises a unit dosage form of a pharmaceutical composition comprising GC-C receptor agonist polypeptide, wherein the assay value for GC-C receptor agonist polypeptide determined on a weight/weight basis decreases by less than 15% or decreases by less than 10% after 18 months or 24 months of storage of the unit dosage form at 25° C. at 60% relative humidity in a sealed container containing a desiccant. In a further embodiment, the assay value for GC-C receptor agonist polypeptide determined on a weight/weight basis decreases by less than 9%, 8%, 7%, 6%, 5%, 4%, 3%, 2% or 1% after 18 months or 24 months of storage of the unit dosage form at 25° C. at 60% relative humidity in a sealed container containing a desiccant. In another embodiment, the invention comprises a unit dosage form of a pharmaceutical composition comprising GC-C receptor agonist polypeptide, wherein the assay value for GC-C receptor agonist polypeptide determined on a weight/weight basis decreases by less than 15% or decreases by less than 10% after 3 months or 6 months of storage of the unit dosage form at 40° C. at 75% relative humidity in a sealed container containing a desiccant. In a further embodiment, the assay value for GC-C receptor agonist polypeptide determined on a weight/weight basis decreases by less than 9%, 8%, 7%, 6%, 5%, 4%, 3%, 2% or 1% after 3 months or 6 months of storage of the unit dosage form at 40° C. at 75% relative humidity in a sealed container containing a desiccant.

In one embodiment, the invention comprises a sealed container comprising a plurality of unit dosage forms of a pharmaceutical composition comprising GC-C receptor agonist polypeptide, wherein the assay value for GC-C receptor agonist polypeptide determined on a weight/weight basis decreases by less than 15% or decreases by less than 10% after 18 months or 24 months of storage of the sealed container at 25° C. at 60% relative humidity in a sealed container containing a desiccant. In a further embodiment, the assay value for GC-C receptor agonist polypeptide determined on a weight/weight basis decreases by less than 9%, 8%, 7%, 6%, 5%, 4%, 3%, 2% or 1% after 18 months or 24 months of storage of the sealed container containing a desiccant at 25° C. at 60% relative humidity. In another embodiment, the invention comprises a sealed container comprising a plurality of unit dosage forms of a pharmaceutical composition comprising GC-C receptor agonist polypeptide, wherein the assay value for GC-C receptor agonist polypeptide determined on a weight/weight basis decreases by less than 15% or decreases by less than 10% after 3 months or 6 months of storage of the sealed container containing a desiccant at 40° C. at 75% relative humidity. In a further embodiment, the assay value for GC-C receptor agonist polypeptide determined on a weight/weight basis decreases by less than 9%, 8%, 7%, 6%, 5%, 4%, 3%, 2% or 1% after 3 months or 6 months of storage of the sealed container containing a desiccant at 40° C. at 75% relative humidity.

The assay value on a weight/weight basis (“weight/weight assay”) may be determined by comparing, e.g., by HPLC, the amount of GC-C receptor agonist polypeptide in a sample, to a GC-C receptor agonist polypeptide reference standard. As used herein, the weight of GC-C receptor agonist polypeptide in a composition after storage at room temperature or accelerated conditions at a specified time point (e.g., three or six months of storage under accelerated conditions [40° C./75% RH] or 12, 18 or 24 months of storage under room temperature conditions [25 ° C./60% RH]) is compared to the weight of GC-C receptor agonist polypeptide in a composition at an initial time (e.g., the time when the pharmaceutical composition is released for clinical or patient use (“the release date”)) to provide the weight/weight assay value. For example, the weight of GC-C receptor agonist polypeptide in a composition is measured after storage for a specified time at accelerated conditions (40° C./75% RH) and compared to the weight of GC-C receptor agonist polypeptide that was present in the sample at the release date. In another example, the weight of GC-C receptor agonist polypeptide in a composition is measured after storage for a specified time at room temperature conditions (25° C./60% RH) and compared to the weight of GC-C receptor agonist polypeptide that was present in the sample at the release date. Thus, the phrase “≧90% of the original amount of GC-C receptor agonist polypeptide in the composition remains after at least 6 months when packaged samples are stored at accelerated conditions (40° C./75% RH)” means the weight of GC-C receptor agonist polypeptide in the composition measured in an assay on a weight/weight basis as determined by HPLC after at least 6 months storage at accelerated conditions is ≧90% of the amount of GC-C receptor agonist polypeptide in the composition present at the initial time (e.g., the release date of the GC-C receptor agonist polypeptide composition).

Chromatographic purity of GC-C receptor agonist polypeptide may be assessed by performing HPLC under the conditions described herein. The area under the GC-C receptor agonist polypeptide peak is measured and compared to the total area under all peaks excluding the solvent peak and any non-polypeptide related peaks (i.e., peaks associated with excipients that may be observed in a placebo). As used herein, the chromatographic purity of GC-C receptor agonist polypeptide in a composition after storage at room temperature or accelerated conditions at a specified time point (e.g., three or six months of storage under accelerated conditions [40° C./75% RH] or 12, 18 or 24 months of storage under room temperature conditions [25 ° C./60% RH]) is compared to the chromatographic purity of GC-C receptor agonist polypeptide in a composition at an initial time (e.g., the time when the pharmaceutical composition is released for clinical or patient use (“the release date”)) to provide the chromatographic purity value. For example, the chromatographic purity of GC-C receptor agonist polypeptide in a composition is measured after storage for a specified time at accelerated conditions (40° C./75% RH) and compared to the chromatographic purity of GC-C receptor agonist polypeptide in the composition at the release date. In another example, the chromatographic purity of GC-C receptor agonist polypeptide in a composition is measured after storage for a specified time at room temperature conditions (25° C./60% RH) and compared to the chromatographic purity of GC-C receptor agonist polypeptide in the composition at the release date.

This disclosure features a method for preparing a pharmaceutical composition comprising GC-C receptor agonist polypeptide or a pharmaceutically acceptable salt thereof, the method comprising: (a) providing a solution, e.g., an aqueous solution (“the coating solution”), comprising: (i) GC-C receptor agonist polypeptide or a pharmaceutically acceptable salt thereof; (ii) a cation selected from Mg²⁺, Ca²⁺, Zn²⁺, Mn²⁺, K⁺, Na⁺or Al⁺ and/or a sterically hindered primary amine (e.g., leucine) and, optionally, (iii) a pharmaceutically acceptable binder; and (b) applying the coating solution to a pharmaceutically acceptable filler to generate polypeptide-coated filler (e.g., by spraying, mixing or coating the pharmaceutically acceptable filler with the coating solution). The method can optionally include one or more of: (i) blending the polypeptide-coated filler with a pharmaceutically acceptable glidant, a pharmaceutically acceptable lubricant or a pharmaceutically acceptable additive that acts as both a glidant and lubricant; (ii) blending the polypeptide-coated filler with filler that is not polypeptide-coated, (iii) blending the polypeptide-coated filler with other additives; (iii) applying a pharmaceutically acceptable coating additive to the polypeptide-coated filler. The final pharmaceutical composition can be placed into capsules (e.g., gelatin capsule) or used to form tablets.

### DETAILED DESCRIPTION

In certain embodiments, the GC-C receptor agonist polypeptide comprises of an amino acid sequence selected from: CCEFCCNPACTGCY (SEQ ID NO: 1), CCEFCCNPACTGC (SEQ ID NO: 2), CCEICCNPACTGCY (SEQ ID NO: 3), CCEICCNPACTGC (SEQ ID NO: 4), CCELCCNPACTGCY (SEQ ID NO: 5), CCELCCNPACTGC (SEQ ID NO: 6), CCEWCCNPACTGCY (SEQ ID NO: 7), CCEWCCNPACTGC (SEQ ID NO: 8), CCEYCCNPACTGC (SEQ ID NO: 9), PGTCEICAYAACTGC (SEQ ID NO: 10), NDDCELCVNVACTGCL (SEQ ID NO: 11) and NDECELCVNVACTGCL (SEQ ID NO: 12). In certain embodiments the GC-C receptor agonist polypeptide does not comprise or consist of the amino acid sequence CCEYCCNPACTGCY (SEQ ID NO: 13). In cetain embodiments, the GC-C receptor agonist polypeptide may be a mixture of two or more GC-C receptor agonist polypeptides described herein.

Oral compositions containing GC-C receptor agonist polypeptide can be used to treat a variety of gastrointestinal disorders. In various embodiments, the patient is suffering from a gastrointestinal disorder; the patient is suffering from a disorder selected from the group consisting of: gastrointestinal motility disorders, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, irritable bowel syndrome, post-operative ileus, ulcerative colitis, chronic constipation, constipation, pain associated with constipation, and disorders and conditions associated with constipation (e.g. constipation associated with use of opiate pain killers, post-surgical constipation, and constipation associated with neuropathic disorders as well as other conditions and disorders described herein); the patient is suffering from a gastrointestinal motility disorder, chronic intestinal pseudo-obstruction, colonic pseudo-obstruction, Crohn's disease, duodenogastric reflux, dyspepsia, functional dyspepsia, nonulcer dyspepsia, a functional gastrointestinal disorder, functional heartburn, gastroesophageal reflux disease (GERD), gastroparesis, inflammatory bowel disease, irritable bowel syndrome (e.g. diarrhea-predominant irritable bowel syndrome (d-IBS), constipation-predominant irritable bowel syndrome (c-IBS) and/or alternating irritable bowel syndrome (a-IBS)), post-operative ileus, ulcerative colitis, chronic constipation, constipation, pain associated with constipation, and disorders and conditions associated with constipation (e.g. constipation associated with use of opiate pain killers, post-surgical constipation, and constipation associated with neuropathic disorders as well as other conditions and disorders described herein); the patient has been diagnosed with a functional gastrointestinal disorder according to the Rome Criteria (e.g. Rome II), the patient has been diagnosed with irritable bowel syndrome (e.g. (e.g. diarrhea predominant-IBS, constipation predominant-IBS, and/or alternating-IBS), according to the Rome Criteria (e.g. Rome II).

The dose range of GC-C receptor agonist polypeptide for adult humans is generally from 25 μg to 6 mg per day orally. In a further embodiment, the dose range is 25 μg to 2 mg per day orally. In some embodiments, the dose range for adult humans is 50 μg to 1 mg per day orally (e.g., 50 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg or 1 mg). In further embodiments, the dose range is 100 μg to 600 μg per day orally. In other embodiments, the dose is 50 μg, 100 μg, 150 μg, 200 μg, 300 μg, 400 μg, 500 μg or 600 μg GC-C receptor agonist polypeptide per day orally. In one embodiment, the GC-C receptor agonist polypeptide composition is provided in a discrete unit, a unit dosage form, (e.g., a tablet, a capsule, a sachet) that is effective at such dosage or as a multiple of the same. In certain embodiments, the unit dosage form and daily dose are equivalent. In various embodiments, the unit dosage form is administered with food at anytime of the day, without food at anytime of the day, with food after an overnight fast (e.g. with breakfast). In various embodiments, the unit dosage form is administered once a day, twice a day or three times a day. The unit dosage form can optionally comprise other additives. In some embodiments, one, two or three unit dosage forms will contain the daily oral dose of GC-C receptor agonist polypeptide. The precise amount of compound administered to a patient will be the responsibility of the attendant physician. However, the dose employed will depend on a number of factors, including the age and sex of the patient, the precise disorder being treated, and its severity.

A cation of the invention may be provided as a pharmaceutically acceptable salt i.e., a cation with an appropriate counterion. Examples of appropriate salts include, without limitation, magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, manganese acetate, manganese chloride, manganese phosphate, manganese sulfate, potassium acetate, potassium chloride, potassium phosphate, potassium sulfate, sodium acetate, sodium chloride, sodium phosphate, sodium sulfate, aluminum acetate, aluminum chloride, aluminum phosphate or aluminum sulfate. In further embodiments, the cation is provided as magnesium chloride, calcium chloride, calcium phosphate, calcium sulfate, zinc acetate, manganese chloride, potassium chloride, sodium chloride or aluminum chloride. In other embodiments, the cation is provided as calcium chloride, magnesium chloride or zinc acetate.

As used herein, the sterically hindered primary amine has the formula:

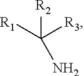

wherein R₁, R₂ and R₃ are independently selected from: H; —C(O)OH; C1-C6 alkyl, optionally substituted by —CO₂H, —CONH₂, or a 5-10 membered aryl or heteroaryl; C1-C6 alkoxyalkyl; or C1-C6 thioalkoxyalkyl, wherein any of the alkyl or aryl groups above can be singly or multiply substituted with halogen or —NH₂, and provided that no more than two of R₁, R₂ and R₃ are H. In a further embodiment, no more than one of R₁, R₂ and R₃ is H.

The term “alkyl”, as used herein, refers to a saturated linear or branched-chain monovalent hydrocarbon radical. Unless otherwise specified, an alkyl group contains 1-20 carbon atoms (e.g., 1-20 carbon atoms, 1-10 carbon atoms, 1-8 carbon atoms, 1-6 carbon atoms, 1-4 carbon atoms or 1-3 carbon atoms). Examples of alkyl groups include, but are not limited to, methyl, ethyl, n-propyl, isopropyl, n-butyl, isobutyl, s-butyl, t-butyl, pentyl, hexyl, heptyl, octyl and the like.

The terms Cₙ₋ₘ,“alkoxyalkyl” and Cₙ₋ₘ “thioalkoxyalkyl” mean alkyl, substituted with one or more alkoxy or thioalkoxy groups, as the case may be, wherein the combined total number of carbons of the alkyl and alkoxy groups, or alkyl and thioalkoxy groups, combined, as the case may be, is between the values of n and m. For example, a C₄₋₆ alkoxyalkyl has a total of 4-6 carbons divided between the alkyl and alkoxy portion; e.g. it can be —CH₂OCH₂CH₂CH₃, —CH₂CH₂OCH₂CH₃ or —CH₂CH₂CH₂OCH₃.

As used herein, the term “aryl” (as in “aryl ring” or “aryl group”), used alone or as part of a larger moiety, refers to a carbocyclic ring system wherein at least one ring in the system is aromatic and has a single point of attachment to the rest of the molecule. Unless otherwise specified, an aryl group may be monocyclic, bicyclic or tricyclic and contain 6-18 ring members. Examples of aryl rings include, but are not limited to, phenyl, naphthyl, indanyl, indenyl, tetralin, fluorenyl, and anthracenyl.

The term “heteroaryl” (or “heteroaromatic” or “heteroaryl group” or “aromatic heterocycle”) used alone or as part of a larger moiety as in “heteroaralkyl” or “heteroarylalkoxy” refers to a ring system wherein at least one ring in the system is aromatic and contains one or more heteroatoms, wherein each ring in the system contains 3 to 7 ring members and which has a single point of attachment to the rest of the molecule. Unless otherwise specified, a heteroaryl ring system may be monocyclic, bicyclic or tricyclic and have a total of five to fourteen ring members. In one embodiment, all rings in a heteroaryl system are aromatic. Also included in this definition are heteroaryl radicals where the heteroaryl ring is fused with one or more aromatic or non-aromatic carbocyclic or heterocyclic rings, or combinations thereof, as long as the radical or point of attachment is in the heteroaryl ring. Bicyclic 6,5 heteroaromatic system, as used herein, for example, is a six membered heteroaromatic ring fused to a second five membered ring wherein the radical or point of attachment is on the six membered ring.

Heteroaryl rings include, but are not limited to the following monocycles: 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, benzopyrazinyl, benzopyranonyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, the term “binder” refers to any pharmaceutically acceptable binder that may be used in the practice of the invention. Examples of pharmaceutically acceptable binders include, without limitation, a starch (e.g., corn starch, potato starch and pre-gelatinized starch (e.g., STARCH 1500® and STARCH 1500 LM®, sold by Colorcon, Ltd.) and other starches), maltodextrin, gelatin, natural and synthetic gums such as acacia, powdered tragacanth, guar gum, cellulose and its derivatives (e.g., methylcellulose, hydroxyethyl cellulose, hydroxyethyl methylcellulose, hydroxypropyl cellulose and hydroxypropyl methylcellulose (hypromellose), ethyl cellulose, cellulose acetate, carboxymethyl cellulose calcium, sodium carboxymethyl cellulose, carboxymethylcellulose, microcrystalline cellulose (e.g. AVICEL™, such as, AVICEL-PH-101™, −103™ and 105™, sold by FMC Corporation, Marcus Hook, Pa., USA)), polyvinyl alcohol, polyvinyl pyrrolidone (e.g., polyvinyl pyrrolidone K30), and mixtures thereof.

As used herein, the term “filler” refers to any pharmaceutically acceptable filler that may be used in the practice of the invention. Examples of pharmaceutically acceptable fillers include, without limitation, talc, calcium carbonate (e.g., granules or powder), dibasic calcium phosphate, tribasic calcium phosphate, calcium sulfate (e.g., granules or powder), microcrystalline cellulose (e.g., Avicel PH101 or Celphere CP-305), powdered cellulose, dextrates, kaolin, mannitol, silicic acid, sorbitol, starch (e.g., Starch 1500), pre-gelatinized starch, lactose, glucose, fructose, galactose, trehalose, sucrose, maltose, isomalt, raffinose, maltitol, melezitose, stachyose, lactitol, palatinite, xylitol, myoinositol, and mixtures thereof. Examples of pharmaceutically acceptable fillers that may be particularly used for coating with GC-C receptor agonist polypeptide include, without limitation, talc, microcrystalline cellulose (e.g., Avicel PH101 or Celphere CP-305), powdered cellulose, dextrates, kaolin, mannitol, silicic acid, sorbitol, starch, pre-gelatinized starch, lactose, glucose, fructose, galactose, trehalose, sucrose, maltose, isomalt, dibasic calcium phosphate, raffinose, maltitol, melezitose, stachyose, lactitol, palatinite, xylitol, mannitol, myoinositol, and mixtures thereof.

As used herein, the term “additives” refers to any pharmaceutically acceptable additive. Pharmaceutically acceptable additives include, without limitation, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, an “excipient” is any pharmaceutically acceptable additive, filler, binder or agent.

As used herein, “purified GC-C receptor agonist polypeptide” is GC-C receptor agonist polypeptide or a pharmaceutically acceptable salt thereof that is greater than or equal to 90 percent pure or greater than or equal to 95 percent pure. GC-C receptor agonist polypeptide purity can be measured, for example, by chromatographic purity of GC-C receptor agonist polypeptide using HPLC. In some embodiments, a GC-C receptor agonist polypeptide may be purified.

In one aspect, the pharmaceutical composition may be prepared by spraying a solution comprising a GC-C receptor agonist polypeptide or a pharmaceutically acceptable salt thereof, on a pharmaceutically acceptable filler to generate polypeptide-coated filler. In one embodiment, the method comprises: (a) providing a solution, e.g., an aqueous solution (“the coating solution”), comprising: (i) a GC-C receptor agonist polypeptide or a pharmaceutically acceptable salt thereof; (ii) a cation selected from Mg²⁺, Ca²⁺, Zn²⁺, Mn²⁺, K⁺, Na or Al³⁺ and/or a sterically hindered primary amine (e.g., leucine) and, optionally, (iii) a pharmaceutically acceptable binder; and (b) applying the coating solution to a pharmaceutically acceptable filler to generate polypeptide-coated filler (e.g., by spraying, mixing or coating the pharmaceutically acceptable filler with the coating solution). The method can optionally include one or more of: (i) blending the polypeptide-coated filler with a pharmaceutically acceptable glidant, a pharmaceutically acceptable lubricant or a pharmaceutically acceptable additive that acts as both a glidant and lubricant; (ii) blending the polypeptide-coated filler with filler that is not polypeptide-coated, (iii) blending the polypeptide-coated filler with other additives; and (iv) applying a pharmaceutically acceptable coating additive to the polypeptide-coated filler. The final pharmaceutical composition can be placed into capsules (e.g., gelatin capsule) or used to form tablets.

In another embodiment, the pharmaceutical composition is prepared by spray drying, which is a technique used to prepare microparticles (e.g., microcapsules or microspheres) of drugs. Spray-dried peptides generally retain their biological activity upon dissolution and may have useful physical characteristics, including a uniform particle size and a spherical shape. In addition, the microparticles prepared by spray drying are often free flowing, which is helpful for pharmaceutical manufacturing processes such as forming tablets and filling capsules. Spray drying processes are also useful because they may be readily scaled up for clinical and commercial manufacturing.

Thus, this disclosure features a method for preparing a pharmaceutical composition comprising a GC-C receptor agonist polypeptide or a pharmaceutically acceptable salt thereof, the method comprising: (a) providing a solution, e.g., an aqueous or organic solution, comprising: (i) a GC-C receptor agonist polypeptide or a pharmaceutically acceptable salt thereof; and (ii) a cation selected from Mg²⁺, Ca²⁺, Zn²⁺, Mn²⁺, K⁺, Na⁺ or Al³⁺ and/or a sterically hindered primary amine (e.g., leucine) and (b) spray drying the polypeptide-containing solution to produce microparticles. The polypeptide-containing solution can optionally include a polymer, such as one or more of the binders described herein, a lipid or phospholipid, and/or a filler, such as mannitol. The method can optionally include one or more additional steps of: (i) blending the polypeptide microparticles with a pharmaceutically acceptable glidant, a pharmaceutically acceptable lubricant or a pharmaceutically acceptable additive that acts as both a glidant and lubricant; (ii) blending the microparticles with a filler, and/or (iii) blending the microparticles with other additives. The final pharmaceutical composition can be placed into capsules (e.g., gelatin capsule) or used to form tablets.

In other embodiments, the pharmaceutical composition is prepared by spray freeze drying, supercritical fluid processing or lyophilization of a solution, e.g., an aqueous or organic solution, comprising: (i) a GC-C receptor agonist polypeptide or a pharmaceutically acceptable salt thereof; and (ii) a cation selected from Mg²⁺, Ca²⁺, Zn²⁺, Mn²⁺, K⁺, Na⁺ or Al³⁺ and/or a sterically hindered primary amine (e.g., leucine).

In some embodiments, the GC-C receptor agonist polypeptide composition is provided in a solid form for oral administration. Examples of such forms include, without limitation, a tablet, a sachet, a pellet, a capsule or a powder. In some embodiments, the compositions can be used to create unit dosages forms, e.g., tablets, capsules, sachets or pellets. Orally administered compositions can include, for example, binders, lubricants, inert diluents, lubricating, surface active or dispersing additives, flavoring additives, and humectants. Orally administered formulations such as tablets may optionally be coated or scored and may be formulated so as to provide sustained, delayed or controlled release of the GC-C receptor agonist polypeptide therein. The GC-C receptor agonist polypeptide can be co-administered or co-formulated with other medications. In one embodiment, the GC-C receptor agonist polypeptide composition can be co-administered with other medications used to treat gastrointestinal disorders. The GC-C receptor agonist polypeptide composition can also be used for treatment of disorders outside the gastrointestinal tract such as congestive heart failure and benign prostatic hypertrophy.

The compositions can include, for example, various additional solvents, dispersants, coatings, absorption promoting additives, controlled release additives, and one or more inert additives (which include, for example, starches, polyols, granulating additives, microcrystalline cellulose, diluents, lubricants, binders, disintegrating additives, and the like), etc. If desired, tablet dosages of the disclosed compositions may be coated by standard aqueous or non-aqueous techniques. Compositions can also include, for example, anti-caking additives, preservatives, sweetening additives, colorants, flavors, desiccants, plasticizers, dyes, and the like.

Suitable disintegrants include, for example, agar-agar, calcium carbonate, microcrystalline cellulose, croscarmellose sodium, crospovidone, povidone, polacrilin potassium, sodium starch glycolate, potato or tapioca starch, other starches, pre-gelatinized starch, clays, other algins, other celluloses, gums, and mixtures thereof.

Suitable lubricants include, for example, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Evonik Degussa Co., Plano, Tex. USA), a pyrogenic silicon dioxide (CAB-O-SIL, Cabot Co., Boston, Mass. USA), and mixtures thereof.

Suitable glidants include, for example, leucine, colloidal silicon dioxide, magnesium trisilicate, powdered cellulose, starch, talc, and tribasic calcium phosphate.

Suitable anti-caking additives include, for example, calcium silicate, magnesium silicate, silicon dioxide, colloidal silicon dioxide, talc, and mixtures thereof.

Suitable anti-microbial additives that may be used, e.g., as a preservative for the GC-C receptor agonist polypeptide compositions, include, for example, benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, and mixtures thereof.

Suitable coating additives include, for example, sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, and mixtures thereof. Suitable protective coatings include Aquacoat (e.g. Aquacoat Ethylcellulose Aquaeous Dispersion, 15% w/w, FMC Biopolymer, ECD-30), Eudragit (e.g. Eudragit E PO PE-EL, Roehm Pharma Polymers) and Opadry (e.g Opadry AMB dispersion, 20% w/w, Colorcon).

In certain embodiments, suitable additives for the GC-C receptor agonist polypeptide composition include one or more of sucrose, talc, magnesium stearate, crospovidone or BHA.

In certain embodiments, the term “95%” may be 95.0%, the term “90%” may be 90.0%, the term “10%” may be 10.0%, the term “9%” may be 9.0%, the term “8%” may be 8.0%, the term “7%” may be 7.0%, the term “6%” may be 6.0%, the term “5%” may be 5.0%, the term “4%” may be 4.0%, the term “3%” may be 3.0%, the term “2%” may be 2.0%, and the term “1%” may be 1.0%.

In certain embodiments, the GC-C receptor agonist polypeptide composition is provided in a unit dosage form. In some embodiments, the unit dosage form is a capsule, a tablet, a sachet, a pellet or a powder. In one such embodiment, the unit dosage form is a capsule or tablet. Such unit dosage forms may be contained in a container such as, without limitation, a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to provide a single dosage form. For example, tablets or capsules may be contained in a bottle which is in turn contained within a box. In some embodiments, the unit dosage forms are provided in a container further comprising a desiccant. In a further embodiment, the unit dosage forms, e.g., a quantity of tablets or capsules, are provided in a container, e.g., a bottle, jar or re-sealable bag, containing a desiccant. In a further embodiment, the container containing the unit dosage forms is packaged with administration or dosage instructions. In certain embodiments, the GC-C receptor agonist polypeptide composition is provided in a kit. The GC-C receptor agonist polypeptide composition described herein and combination therapy agents can be packaged as a kit that includes single or multiple doses of two or more agents, each packaged or formulated individually, or single or multiple doses of two or more agents packaged or formulated in combination. Thus, the GC-C receptor agonist polypeptide composition can be present in first container, and the kit can optionally include one or more agents in a second container. The container or containers are placed within a package, and the package can optionally include administration or dosage instructions.

### EXAMPLES

GC-C receptor agonist polypeptide or a pharmaceutically acceptable salt thereof may be produced and purified using standard techniques known in the art, e.g., chemical synthesis or recombinant expression followed by and purification using standard techniques.

### Example 1

### Formulation Method A

Preparation of the Coating Solution: Approximately 32 g to 42 g of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The pH of the solution is tested, and hydrochloric acid is added, if necessary, to produce a solution having a pH between 1.5 and 2.0. The binder is then added to the solution and the mixture is then stirred for sufficient time to achieve a clear solution. The desired amount of GC-C receptor agonist polypeptide is added to the solution and mixed for 30-100 minutes to provide the coating solution.

Preparation of the Active Beads: Approximately 30-36 g of dried microcrystalline cellulose beads are added to a Mini Column Fluid Bed Coater. The microcrystalline cellulose beads are fluidized and heated prior to layering. Next, the coating solution is layered to the beads. The spraying temperature is controlled between 24° C. and 55° C. by controlling inlet temperature, spray rate, atomization pressure, and air volume. After the entire coating solution is layered to the beads, the beads are dried. The product of this process is referred to as active beads.

Preparation of Active Beads with Protective Coating: Approximately 35 g of Active Beads are added to a Mini Column Fluid Bed Coater. The Active Beads are fluidized and heated prior to coating with Aquacoat (e.g. Aquacoat Ethylcellulose Aquaeous Dispersion, 15% w/w, FMC Biopolymer, ECD-30), Eudragit (e.g. Eudragit E PO PE-EL, Roehm Pharma Polymers) or Opadry (e.g Opadry AMB dispersion, 20% w/w, Colorcon). Next, the coating solution is layered to the beads. The spraying temperature is controlled between 24° C. and 55° C. by controlling inlet temperature, spray rate, atomization pressure, and air volume. After the entire coating solution is layered to the beads, the beads are dried.

### Example 2

### Formulation Method B

Preparation of the Coating Solution: Approximately 8.3 kg of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The binder is then added to the solution and the solution is mixed for sufficient time to achieve a clear solution. The pH of the solution is tested, and hydrochloric acid is added if necessary to produce a solution having a pH between 1.5 and 2.0. This is Solution 1. Approximately 8.3 kg of purified water is mixed with hydrochloric acid to create a solution with a pH between 1.5 and 2.0. The desired amount of GC-C receptor agonist polypeptide is added to the solution and mixed for 10 to 30 minutes. The pH of the solution is tested, and hydrochloric acid is added if necessary to produce a solution having a pH between 1.5 and 2.0. This is Solution 2. Solution 1 and Solution 2 are then mixed together. The pH of the solution is tested, and hydrochloric acid is added if necessary to produce a solution having a pH between 1.5 and 2.0. This is the coating solution.

Preparation of the Active Beads: Approximately 24.19 kg of microcrystalline cellulose beads are added to a Wurster Column of a Glatt GPCG-30 Fluid Bed. The microcrystalline cellulose beads are fluidized and heated to product temperature of 45-47° C.

Next, the coating solution is layered to the beads. The product spraying temperature is controlled between 37° C. and 47° C. by controlling inlet temperature, spray rate, atomization pressure, and air volume. After the entire coating solution is layered to the beads, the beads are dried with a product drying temperature of 37° C. to 47° C. The product of this process is referred to as active beads.

### Example 3

### Preparation of Capsules Containing GC-C Receptor Agonist Polypeptide Formulation

The GC-C receptor agonist polypeptide content on active beads may be measured as described below or by other equivalent methods.

To form capsules suitable for oral administration, an appropriate amount of active beads is used to fill gelatin capsules (e.g., Size 2 gelatin capsules). An appropriate amount of active beads may contain 50 μg to 2 mg GC-C receptor agonist polypeptide per capsule with a range of ±5%. In some embodiments, the appropriate amount of GC-C receptor agonist polypeptide on active beads may be 50 μg, 100 μg, 150 μg, 200 μg, 300 μg, 400 μg, 500 μg, 600 μg, 700 μg, 800 μg, 900 μg, 1 mg, 2 mg, 4 mg or 6 mg. In a particular embodiment, the appropriate amount of GC-C receptor agonist polypeptide on active beads is 100 μg, 150 μg, 200 μg, 300 μg, 400 μg, 500 μg, 600 μg. In a more particular embodiment, the appropriate amount of GC-C receptor agonist polypeptide on active beads is 150 μg or 300 μg per capsule.

In another embodiment, an appropriate amount of active beads to fill a desired number of gelatin capsules is placed in a container. One or more pharmaceutically acceptable fillers or other pharmaceutically acceptable additives may be added, if desired, to the container. In some embodiments, a filler or additive is talc, leucine, microcrystalline cellulose or mannitol. The contents of the container are blended and the mixture is used to fill gelatin capsules with an appropriate amount of active beads containing GC-C receptor agonist polypeptide (e.g., 50 μg to 2 mg GC-C receptor agonist polypeptide per capsule with a range of ±5%).

In an alternative embodiment, an appropriate amount of active beads is used to fill gelatin capsules and one or more pharmaceutically acceptable fillers or other pharmaceutically acceptable additives are added to the gelatin capsules.

### Example 4

### Measurement of GC-C Receptor Agonist Polypeptide Content and Purity

GC-C receptor agonist polypeptide content and purity, as well as measurement of GC-C receptor agonist polypeptide-related substances may be determined by reverse phase gradient liquid chromatography. For example, HPLC analysis of certain polypeptides can be conducted using an Agilent Series 1100 LC System with Chemstation Rev A.09.03 software or equivalent. A YMC Pro™ C18 column (dimensions: 3.0×150 mm, 3.5 um, 120 Å; Waters Corp., Milford, Mass.) or equivalent is used and is maintained at 40° C. Mobile phase A (MPA) consists of water with 0.1% trifluoroacetic acid while mobile phase B (MPB) consists of 95% acetonitrile:5% water with 0.1% trifluoroacetic acid. Elution of GC-C receptor agonist polypeptide and its related substances is accomplished with a gradient from 0% to 47% MPB in 28 minutes followed by a ramp to 100% MPB in 4 minutes with a 5 minute hold at 100% MPB to wash the column. Re-equilibration of the column is performed by returning to 0% MPB in 1 minute followed by a 10 minute hold at 100% MPA. The flow rate is 0.6 mL/min and detection is accomplished by UV at 220 nm.

Samples for analysis are prepared by addition of the contents of GC-C receptor agonist polypeptide capsules to 0.1 N HCl to obtain a target concentration of 20 μg GC-C receptor agonist polypeptide/mL. 100 μL, of this solution is injected onto the column.

GC-C receptor agonist polypeptide content is measured by determining the GC-C receptor agonist polypeptide concentration in the prepared sample against a similarly prepared external GC-C receptor agonist polypeptide standard.

### Examples 5-6

### Preparation of Formulations and Stability Testing

The polypeptide formulations of Examples 5-6 were produced essentially as described in Example 1. The coating solution contained 0.7% Methocel (hydroxypropyl methyl cellulose) as a binder (w/v), and the coating solution was sprayed on Celphere CP-305 beads as described in Example 1. Table 1 provides the cation and amine along with their molar ratios relative to the GC-C receptor polypeptide:

| | | | | Molar |
|---|---|---|---|---|
| Example | Cation* | Amine | Polypeptide | Ratio |
| 5 | CaCl₂•2H₂O | Leucine | CCEYCCNPACTGC | 60:30:1 |
| | | | SEQ ID NO: 9 | |
| 6 | CaCl₂•2H₂O | Leucine | CCEFCCNPACTGCY | 60:30:1 |
| | | | SEQ ID NO: 1 | |

*“Cation” refers to the cation contained in the salt used in the example, “Amine” refers to the sterically hindered primary amine, “Polypeptide” refers to the GC-C receptor agonist polypeptide; “Molar Ratio” refers to the molar ratio of the cation:amine:polypeptide.

Gelatin capsules were filled with approximately 225 mg of active beads (600 μg polypeptide/225 mg of active beads) for SEQ ID NO: 1 and 225 mg of active beads (150 μg polypeptide/225 of active beads) for SEQ ID NO: 9). Five filled capsules were placed in plastic bottles. The bottles contained 1 g of desiccant and were induction sealed. The bottles were stored at 40° C./75% RH for three months. Polypeptide content and percent chromatographic purity (% CP) were measured at the initial time point and one and three months after storage at 40° C./75% RH. The polypeptide content on a weight/weight basis (“weight/weight assay”) may be determined by comparing, e.g., by HPLC, the amount of polypeptide in a sample, to a reference standard of that polypeptide. Chromatographic purity of a polypeptide may be assessed by performing HPLC. The area under the polypeptide peak is measured and compared to the total area under all peaks excluding the solvent peak and any non-polypeptide related peaks (i.e., peaks associated with excipients that may be observed in a placebo).Results are provided below:

| | | Assay [w/w] | | % CP |
|---|---|---|---|---|
| Example | Time (months) | % of Initial* | % CP | [% of Initial] |
| 5 | 1 | 98.94% | 96.52% | 99.46% |
| 5 | 3 | 90.27% | 93.82% | 96.68% |
| 6 | 1 | 96.01% | 79.19% | 99.90% |
| 6 | 3 | 49.76% | 80.80% | 101.93% |

1-118. (canceled)

119. A pharmaceutical composition comprising a pharmaceutically acceptable carrier, a GC-C receptor agonist polypeptide and one or more agents selected from (i) a cation selected from Mg²⁺, Zn²⁺, Mn²⁺, K⁺, Na⁺ or Al³⁺, or (ii) a sterically hindered primary amine, wherein the agent improves at least one attribute of the composition, relative to a pharmaceutical composition without the agent, after (a) a first 18 months of storage of the pharmaceutical composition at 25° C. at 60% relative humidity in a sealed container containing a desiccant or (b) a first 6 months of storage of the pharmaceutical composition at 40° C. at 75% relative humidity in a sealed container containing a desiccant,
wherein the attribute is selected from a decrease in the rate of degradation of GC-C receptor agonist polypeptide as measured by GC-C receptor agonist polypeptide content, a decrease in the rate of degradation of GC-C receptor agonist polypeptide as measured by chromatographic purity of GC-C receptor agonist polypeptide, a decrease in the amount of a GC-C receptor agonist polypeptide oxidation product relative to the amount of GC-C receptor agonist polypeptide, and a decrease in the amount of a GC-C receptor agonist polypeptide hydrolysis product relative to the amount of GC-C receptor agonist polypeptide.

120. The pharmaceutical composition according to claim 119, wherein the GC-C receptor agonist polypeptide comprises an amino acid sequence selected from the group consisting of: CCEFCCNPACTGCY (SEQ ID NO: 1), CCEFCCNPACTGC (SEQ ID NO: 2), CCEICCNPACTGCY (SEQ ID NO: 3), CCEICCNPACTGC (SEQ ID NO: 4), CCELCCNPACTGCY (SEQ ID NO: 5), CCELCCNPACTGC (SEQ ID NO: 6), CCEWCCNPACTGCY (SEQ ID NO: 7), CCEWCCNPACTGC (SEQ ID NO: 8), PGTCEICAYAACTGC (SEQ ID NO: 10), NDDCELCVNVACTGCL (SEQ ID NO: 11) and NDECELCVNVACTGCL (SEQ ID NO: 12); wherein said polypeptide does not comprise the amino acid of CCEYCCNPACTGCY (SEQ ID NO: 13).

121. The pharmaceutical composition according to claim 119, wherein the GC-C receptor agonist polypeptide consists of an amino acid sequence selected from the group consisting of: CCEFCCNPACTGCY (SEQ ID NO: 1), CCEFCCNPACTGC (SEQ ID NO: 2), CCEICCNPACTGCY (SEQ ID NO: 3), CCEICCNPACTGC (SEQ ID NO: 4), CCELCCNPACTGCY (SEQ ID NO: 5), CCELCCNPACTGC (SEQ ID NO: 6), CCEWCCNPACTGCY (SEQ ID NO: 7), CCEWCCNPACTGC (SEQ ID NO: 8), CCEYCCNPACTGC (SEQ ID NO: 9), PGTCEICAYAACTGC (SEQ ID NO: 10), NDDCELCVNVACTGCL (SEQ ID NO: 11) and NDECELCVNVACTGCL (SEQ ID NO: 12).

122. The pharmaceutical composition according to claim 119, wherein the pharmaceutical composition is prepared by a process comprising the steps:
(a) providing an aqueous solution comprising:
(i) the GC-C receptor agonist polypeptide or a pharmaceutically acceptable salt thereof
(ii) one or more of a cation selected from Mg²⁺, Zn²⁺, Mn²⁺, K⁺, Na⁺ or Al³⁺; or a sterically hindered primary amine; and
(iii) a pharmaceutically acceptable binder; and
(b) applying the aqueous solution to a pharmaceutically acceptable filler to generate GC-C receptor agonist polypeptide-coated filler.

123. The pharmaceutical composition according to claim 122, wherein the aqueous solution is applied to the filler by spraying.

124. The pharmaceutical composition according to claim 123, wherein the aqueous solution comprises a cation and a sterically hindered primary amine.

125. A capsule or tablet comprising the pharmaceutical composition according according to claim 119.

126. The capsule or tablet according to claim 125, wherein each capsule or tablet comprises 50 μg to 1 mg GC-C receptor agonist polypeptide.

127. A unit dosage form comprising the pharmaceutical composition of claim 119.

128. A sealed container comprising a plurality of unit dosage forms according to claim 127.

129. A method for treating a patient suffering from impaired intestinal motility, irritable bowel syndrome, constipation, pain associated with constipation, dyspepsia, gastroparesis or chronic intestinal pseudo obstruction, comprising administering to the patient an effective amount of the pharmaceutical composition according to claim 119.

130. The method according to claim 129, wherein the irritable bowel syndrome is constipation-predominant irritable bowel syndrome or alternating irritable bowel syndrome.

131. The method according to claim 130, wherein the irritable bowel syndrome is constipation-predominant irritable bowel syndrome.

132. The method according to claim 129, wherein the constipation is chronic constipation, idiopathic constipation, post-operative ileus, or constipation caused by opiate use.

133. The method according to claim 132, wherein the constipation is chronic constipation.
